(19) United States
(12) Patent Application Publication
(10) Pub. No.: US 2023/0166085 A1
(43) Pub. Date: Jun. 1, 2023

(54) Split Septum Needle Free Connector Alignment and Flushing Features

(71) Applicant: Becton, Dickinson and Company, Franklin, NJ (US)

(72) Inventors: Erica E. Neumann, Salt Lake City, UT (US); John M. Lackey, West Valley City, UT (US); Jonathan Karl Burkholz, Salt Lake City, UT (US)

(21) Appl. No.: 17/994,920

(22) Filed: Nov. 28, 2022

Related U.S. Application Data
(60) Provisional application No. 63/284,118, filed on Nov. 30, 2021.

(51) Int. Cl. A61M 25/06; A61B 5/15

(52) U.S. Cl. CPC A61M 25/0606; A61B 5/15003

(57) ABSTRACT

An integrated intravenous catheter including a catheter adapter having a catheter and an inlet, the catheter configured to be inserted into a patient's vasculature, as well as a needle free connector. The needle free connector includes first port, a second port positioned opposite the first port, and a side port positioned between the first port and the second port. The needle free connector further includes a main cavity portion having a tapered sidewall and a distal taper portion, wherein the tapered sidewall gradually narrows in a direction of the distal taper portion. The integrated intravenous catheter also includes intermediate tubing extending between the inlet of the catheter adapter and the first port of the needle free connector, and extension tubing extending from the side port of the needle free connector.

### CROSS-REFERENCE TO RELATED APPLICATION

The present application claims priority to U.S. Provisional Patent Application Ser. No. 63/284,118, entitled “Split Septum Needle Free Connector Alignment and Flushing Features” filed Nov. 30, 2021, the entire disclosure of which is hereby incorporated by reference in its' entirety.

### BACKGROUND OF THE INVENTION

### Field of the Invention

The present disclosure relates to an integrated intravenous catheter with a needle free connector (NFC) configured for use with a blood draw device.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (“PIVC”). The over-the-needle catheter may be mounted over an introducer needle having a sharp distal tip. The catheter and the introducer needle may be assembled so that the distal tip of the introducer needle extends beyond the distal tip of the catheter with the bevel of the needle facing up away from a skin surface of the patient. The catheter and introducer needle are generally inserted at a shallow angle through the skin into the vasculature of the patient. In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using a peripheral IV catheter may be difficult for several reasons, particularly when an indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, catheters may often be used for acquiring a blood sample at a time of catheter placement but are much less frequently used for acquiring a blood sample during the catheter dwell period.

Accordingly, blood draw devices have been developed to collect blood samples through an existing PIVC. Blood draw devices attach to the PIVC and include a flexible flow tube that is advanced through the PIVC, beyond the catheter tip, and into a vessel to collect a blood sample. After blood collection, the blood draw device is removed from the PIVC and discarded. One example of such a blood draw device, known as PIVO™ from Velano Vascular, Inc., is shown and described in U.S. Pat. No. 11,090,461, which is hereby incorporated by reference in its entirety.

As described in U.S. Pat. No. 11,090,461, the blood draw device includes an introducer having an actuator slidably coupled thereto, with the actuator being configured to selectively advance the flexible flow tube through the PIVC. The introducer is couplable to, e.g., a needle free connector (NFC) of a near-patient access port by way of a lock positioned on a distal end portion of the introducer. The lock may include, e.g., a proboscis (or nose) and a clip comprising a pair of deflectable arms. However, in certain circumstances, the lock may be incorrectly or incompletely coupled to the NFC, potentially leading to misalignment of the nose within the NFC. Such misalignment of the nose within the NFC may result in bowing and/or kinking of the flexible flow tube as it is advanced through the NFC, as the internal structure of the NFC may include “catch points” which can be contacted by the flexible flow tube if misalignment of the lock occurs. The bowing and/or kinking of the flexible flow tube in this way may adversely affect the smooth advancement (or retraction) of the flexible flow tube and/or the collection of blood through the flexible flow tube of the blood draw device.

### SUMMARY OF THE INVENTION

Accordingly, there is a need to provide a needle free connector (NFC) configured to accommodate and correctly align the lock of a blood draw device thereon. Additionally, there is a need for a NFC having improved flushing characteristics.

In accordance with an aspect of the present disclosure, an integrated intravenous catheter is disclosed, the integrated intravenous catheter including a catheter adapter having a catheter and an inlet, the catheter configured to be inserted into a patient's vasculature, and a needle free connector having a first port, a second port positioned opposite the first port, and a side port positioned between the first port and the second port, wherein the needle free connector further includes a main cavity portion having a tapered sidewall and a distal taper portion, and wherein the tapered sidewall gradually narrows in a direction of the distal taper portion. The integrated intravenous catheter also includes intermediate tubing extending between the inlet of the catheter adapter and the first port of the needle free connector, and extension tubing extending from the side port of the needle free connector.

In some embodiments, the needle free connector includes a body defining a flow path extending between the first port and the second port.

In some embodiments, the side port is offset from a center of the flow path.

In some embodiments, the side port is not offset from a center of the flow path.

In some embodiments, the body of the needle free connector further includes a lip portion extending 360° around the needle free connector, and the lip portion is configured to provide a latching surface for engagement with clip portions of a lock of a blood draw device when coupled to the needle free connector.

In some embodiments, a gap is formed between the lip portion and the side port.

In some embodiments, a medical connector is positioned at an end of the extension tubing.

In some embodiments, the needle free connector includes a body defining a longitudinal axis extending between the first port and the second port, and the side port extends from the body at an angle of 15-165 degrees relative to the longitudinal axis of the body.

In some embodiments, the main cavity portion of the needle free connector includes an internal structure configured to redirect fluid when fluid enters the needle free connector via the side port.

In some embodiments, the side port includes a fluid flow path, and an internal surface of the side port at a distal portion of the fluid flow path includes a fluid directing ramp.

In some embodiments, the fluid directing ramp is positioned on a lower internal surface of the side port and is configured to divert flow upward relative to a central plane of the main cavity portion of the needle free connector.

In some embodiments, the fluid directing ramp is positioned on an upper internal surface of the side port and is configured to divert flow downward relative to a central plane of the main cavity portion of the needle free connector.

In some embodiments, the side port includes a fluid flow path, and wherein a distal portion of the fluid flow path of the side port includes a flow splitter.

According to another aspect of the present disclosure, a needle free connector is disclosed. The needle free connector may include a body, a first port positioned at a first end portion of the body, a second port positioned at a second end portion of the body opposite the first port, and a side port positioned between the first port and the second port. The body includes a main cavity portion positioned between the first port and the second port, the main cavity portion having a tapered sidewall and a distal taper portion, and the tapered sidewall gradually narrows in a direction of the distal taper portion.

In some embodiments, the body further includes a lip portion extending 360° around the needle free connector, and the lip portion is configured to provide a latching surface for engagement with clip portions of a lock of a blood draw device when coupled to the needle free connector.

In some embodiments, a gap is formed between the lip portion and the side port.

In some embodiments, the side port includes a fluid flow path, and an internal surface of the side port at a distal portion of the fluid flow path of the side port includes a fluid directing ramp.

In some embodiment, the fluid directing ramp is positioned on a lower internal surface of the side port and is configured to divert flow upward relative to a central plane of the main cavity portion of the needle free connector.

In some embodiment, the fluid directing ramp is positioned on an upper internal surface of the side port and is configured to divert flow downward relative to a central plane of the main cavity portion of the needle free connector.

In some embodiments, the side port includes a fluid flow path, and wherein a distal portion of the fluid flow path of the side port includes a flow splitter.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of an integrated intravenous catheter in accordance with an aspect of the present disclosure;

FIG. 2 is a perspective view of a blood draw device configured for use with the integrated intravenous catheter of FIG. 1;

FIG. 3 is a side view of a needle free connector in accordance with an aspect of the present disclosure;

FIG. 4 is a cross-sectional side view of the needle free connector of FIG. 3;

FIG. 5 is a side view of the needle free connector of FIG. 3 coupled to a lock of a blood draw device in accordance with an aspect of the present disclosure;

FIG. 6 is a side perspective view of a needle free connector in accordance with another aspect of the present disclosure;

FIG. 7 is an end cross-sectional view of the needle free connector of FIG. 6;

FIG. 8A is an end cross-sectional view of a needle free connector in accordance with an aspect of the present disclosure;

FIG. 8B is an end cross-sectional view of a needle free connector in accordance with another aspect of the present disclosure;

FIG. 8C is an end cross-sectional view of a needle free connector in accordance with another aspect of the present disclosure; and

FIG. 8D is an end cross-sectional view of a needle free connector in accordance with another aspect of the present disclosure.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

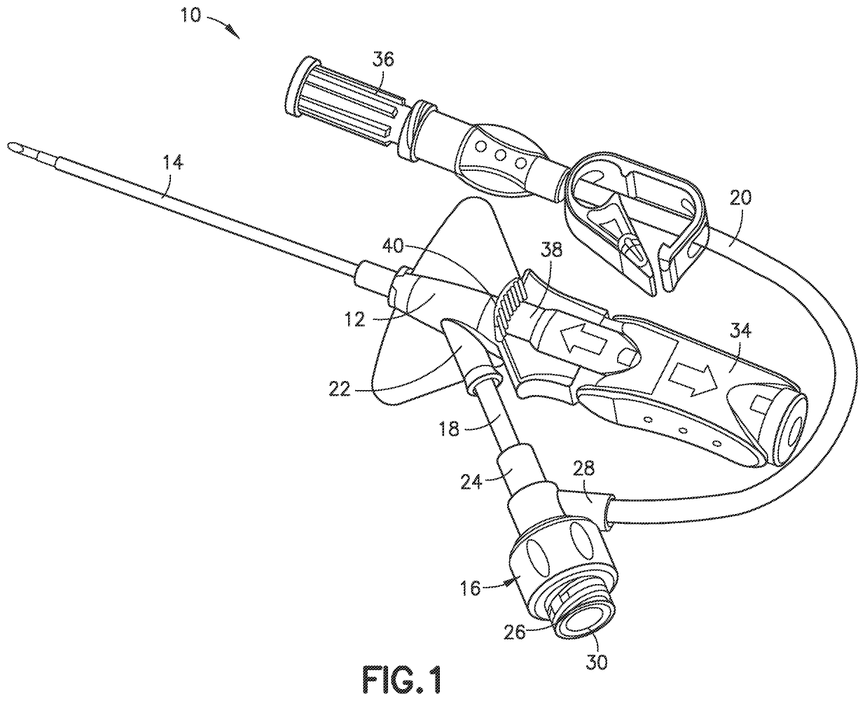

Referring to FIG. 1, an integrated intravenous catheter 10 in accordance with an aspect of the present disclosure is shown. The integrated intravenous catheter 10 includes a catheter adapter 12 having a catheter 14 configured to be inserted into a patient's vasculature, a needle free connector (NFC) 16, intermediate tubing 18, and extension tubing 20. The catheter adapter 12 includes an inlet 22. In the embodiment shown in FIG. 1, the needle free connector 16 includes a first port 24, a second port 26 positioned opposite the first port 24, and a side port 28 positioned between the first port 24 and the second port 26. The second port 26 includes a valve member 30.

The intermediate tubing 18 extends between the inlet 22 of the catheter adapter 12 and the first port 24 of the needle free connector 16. The extension tubing 20 extends from the side port 28 of the needle-free connector 16. The intermediate tubing 18 is configured to provide flexibility when inserting and dressing the catheter 14 and also when manipulating the needle free connector 16 for flushing, blood draw, and/or other procedure without disturbing the catheter insertion site.

Referring still to FIG. 1, in some embodiments, the integrated catheter 10 includes a needle hub assembly 34 and a medical component 36, such as, e.g., a vent plug, with the medical component 36 coupled to the side port 28 of the needle free connector 16 via the extension tubing 20. The needle hub assembly 34 is assembled with the catheter adapter 12 by inserting a needle (not shown) into a lumen of the catheter 14. In one aspect or embodiment, the needle hub assembly 34 includes a needle shield 38 configured to secure a tip of the needle within the needle shield 38 after use. The needle shield 38 may be activated passively. The needle hub assembly 34 includes a push tab 40 to facilitate catheter advancement during insertion. The push tab 40 also allows for one-handed or two-handed advancement. In one aspect or embodiment, the catheter adapter 12 includes one or more wings, as shown, configured to engage a skin surface of a patient. In another aspect or embodiment, the catheter adapter 12 does not include wings.

In some embodiments, at least a portion of the needle free connector 16 is transparent. The connector components of the integrated catheter 10 may be transparent, opaque, and/or colored. In one aspect or embodiment, the needle free connector 16 includes an anti-reflux valve.

In some embodiments, the medical component 36 at the end of the extension tubing 20 is a single port or dual port connector and may include a variety of connectors, including needle free connectors or needle access connectors, such as a PRN. The extension tubing 20 may be left or right facing. In some embodiments, in addition to a vent plug, the medical component 36 may be a removable or non-removable needle free connector or needle access connectors, such as a PRN, that is attached to a female luer connection provided on the extension tubing 20. In some embodiments, a dual female luer port may be bonded or otherwise attached to the extension tubing 20 instead of a single luer connector.

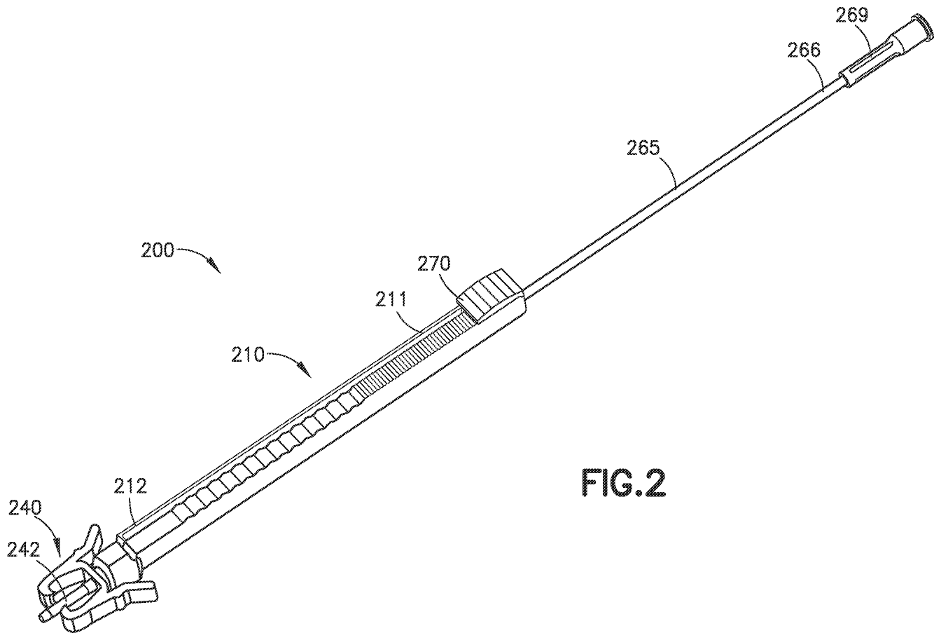

Next, referring to FIG. 2, a blood draw device 200 in accordance with an aspect of the present disclosure is illustrated. The blood draw device 200 may be, e.g., the PIVO™ blood draw device commercially available from Velano Vascular, Inc. In one aspect or embodiment, the blood draw device 200 is the same or similar to the blood draw device shown in U.S. Pat. No. 11,090,461, which is hereby incorporated by reference in its entirety. In one aspect or embodiment, the blood draw device 200 may be any device that advances tubing, a probe, a guidewire, instrument, and/or sensor into the fluid path of the integrated intravenous catheter 10 or beyond the tip of the catheter 14.

Blood draw device 200 may include an introducer 210, a lock 240, a secondary catheter 265, and an actuator 270. The introducer includes a proximal end portion 211 and a distal end portion 212, with the lock 240 being located adjacent the distal end portion 212. The secondary catheter 265 includes the proximal end portion 266 which is coupled to and/or otherwise includes a coupler 269. The coupler 269 is configured to physically and fluidically couple the secondary catheter 265 to any suitable device such as, for example, a fluid reservoir, fluid source, syringe, evacuated container holder (e.g., having a sheathed needle or configured to be coupled to a sheathed needle), pump, and/or the like.

In accordance with some embodiments, a user may manipulate the blood draw device 200 to couple the lock 240 to, e.g., the catheter adapter 12 of integrated intravenous catheter 10. For example, in some embodiments, the user can exert a force sufficient to pivot the first and second clip arms of the lock 240 such that a portion of the catheter adapter 12 can be inserted into the space defined between the arms of the lock 240 and, for example, a nose 242 extending distally from the lock 240. In some embodiments, the nose 242 can be inserted into, e.g., the needle free connector 16 of the catheter adapter 12 when the lock 240 is coupled thereto, while the first and second clip arms of the lock 240 may latch onto an exterior surface (or surfaces) of the needle free connector 16 to hold the blood draw device 200 in place relative to the catheter adapter 12. The nose 242 is sufficiently long to dispose at least a portion of the nose 242 within the needle free connector 16, thereby providing a path for the flexible flow tube to pass from the blood draw device 200 through the catheter adapter 12 of the integrated catheter 10. In some embodiments, the needle free connector 16 is a split septum-type needle free connector.

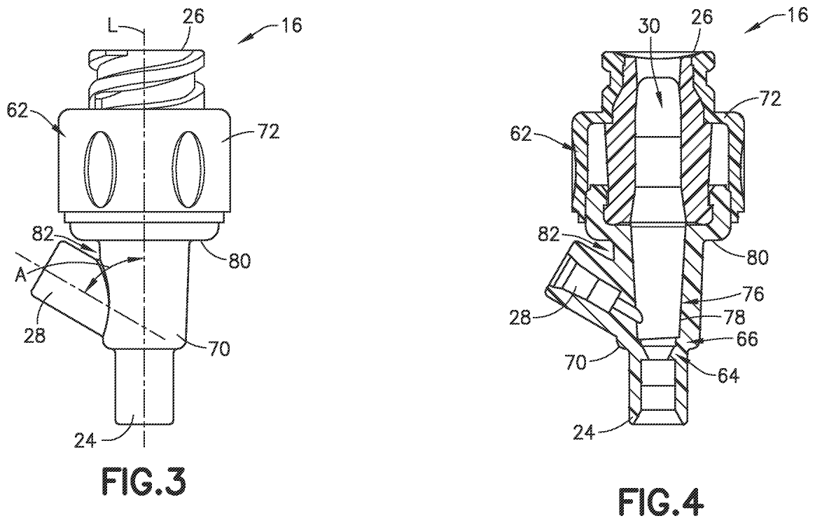

Next, referring to FIGS. 3 and 4, various details of needle free connector 16 in accordance with an aspect of the present disclosure are shown. The needle free connector 16 includes a body 62 defining a flow path 30 extending between the first port 24 and the second port 26. In some embodiments, the side port 28 may be offset from a center of the flow path 30. The offset of the side port 28 is configured to cause fluid entering the body 62 via the side port 28 to enter along an interior surface of the body 62 and cause a vortex or otherwise redirect fluid within the body 62 to aid flushing of the needle free connector 16. In some embodiments, the body 62 of the needle free connector 16 further includes internal structure 66 configured to create a vortex or otherwise redirect fluid when fluid enters the needle free connector 16 via the side port 28. In some embodiments, the offset and vortex-creating feature is the same or similar to the flushing features shown and described in U.S. Patent Application Publication No. 2021/0220548, which is hereby incorporated by reference in its entirety.

The body 62 of the needle free connector 16 defines a longitudinal axis L extending between the first port 24 and the second port 26, with the side port 28 extending from the body 62 at an angle A of e.g., 15°-165° relative to the longitudinal axis L of the body 62. In one embodiment, the side port 28 extends from the body 62 at an angle of 60° relative to the longitudinal axis L of the body 62. The side port 28 extends at the angle A toward the second port 26, although other suitable arrangements may be utilized. The body 62 of the needle free connector 16 includes a first portion 70 and a second portion 72 connected to the first portion 70. In some embodiments, the first portion 70 of the body 62 is fixedly secured to the second portion 72 of the body 62.

Referring to FIG. 4, needle free connector 16 further comprises a main cavity portion 76, wherein main cavity portion 76 includes a tapered sidewall 78. Tapered sidewall 78 is configured to gradually narrow in the direction of first port 24, eventually terminating adjacent a distal taper portion 64. With the gradual taper of tapered sidewall 78 and the distal taper portion 64, the main cavity portion 76 is capable of receiving and constraining the nose (not shown) of a blood draw device such that the nose remains substantially aligned along the longitudinal axis L of the body 62, regardless of the angle of entry of the nose within main cavity portion 76. In this way, the flexible flow tube of the blood draw device may be directed into the distal flow path of the needle free connector 16, thereby avoiding any interference from internal geometry of the main cavity portion 76, etc., due to misalignment of the lock of the blood draw device when coupled to the needle free connector 16, which could cause undesirable bowing and/or kinking of the flexible flow tube.

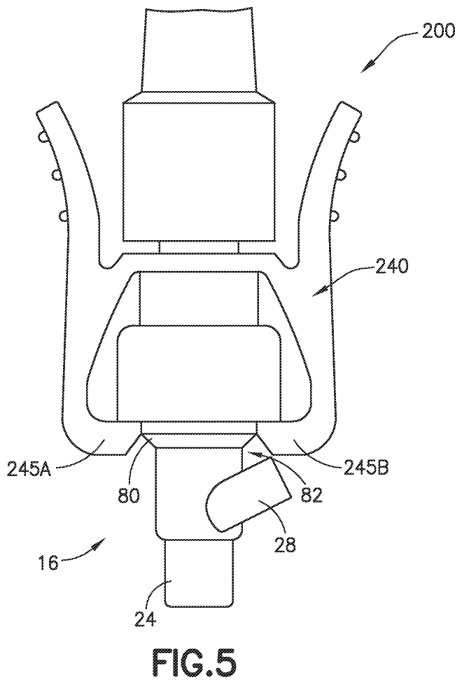

Additionally, referring to FIGS. 3-5, needle free connector 16 further comprises a lip portion 80, with lip portion 80 extending substantially 360° around the needle free connector 16 at or near the interface been first portion 70 and second portion 72. Below the lip portion 80 and above the side port 28, a gap 82 is present. As shown in FIG. 5, the gap 82 is sized and configured such that the respective clip portions 245A, 245B of the lock 240 of blood draw device 200 are capable of securely latching onto the lip portion 80, without interference from the side port 28 in certain orientations. Accordingly, the lock 240 is capable of being coupled to the needle free connector 16 in any orientation, thereby ensuring a secure connection between the blood draw device 200 and the needle free connector 16, and also helping to avoid any potential misalignment of the nose of the blood draw device 200 within the needle free connector 16.

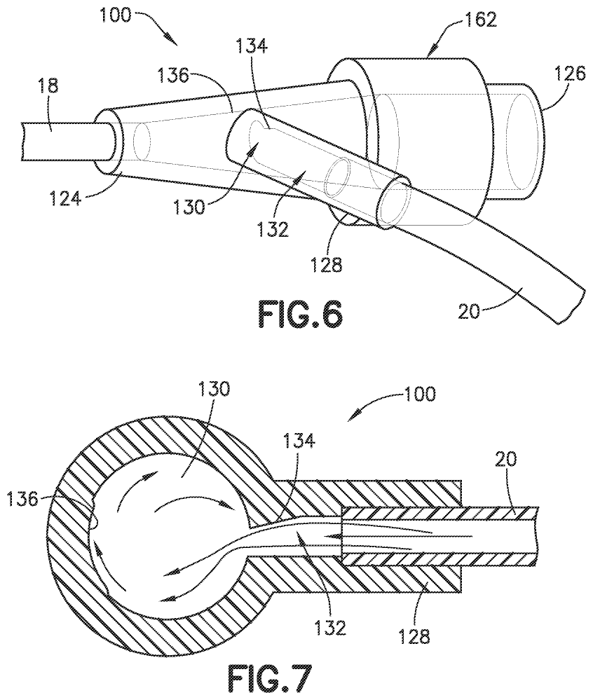

Next, referring to FIGS. 6 and 7, a needle free connector 100 in accordance with another aspect of the present disclosure is illustrated. Similar to needle free connector 16 described above, needle free connector 100 includes a body 162 having a main cavity portion 130 extending between a first port 124 and a second port 126, with the first port 124 being coupled to, e.g., the intermediate tubing 18. Additionally, the needle free connector 100 includes a side port 128, which may be coupled to, e.g., extension tubing 20. In some embodiments, side port 128 is not offset relative to a central axis of the body 162, thereby allowing any parting line formed during manufacturing of the needle free connector 100 to be uniform, simplifying the formation of the needle free connector 100. The main cavity portion 130 may further include a proximal flow diverter 136 configured to cause a vortex or otherwise redirect fluid within the body 162 to aid flushing of the needle free connector 100.

In some embodiments, the side port 128 may be utilized to introduce a flushing fluid into the needle free connector 100. As shown in FIGS. 6 and 7, a distal portion of a fluid flow path 132 of the side port 128 may include a fluid directing ramp 134, which is configured to direct fluid from the fluid flow path 132 toward the proximal flow diverter 136, promoting a swirling effect to aid in flushing a septum of the needle free connector 100. As side port 128 is not offset, the fluid directing ramp 134 provides the necessary flow diversion to promote this swirling effect. The fluid directing ramp 134 may be provided on a single side of the side port 128, immediately before the fluid enters the main cavity portion 130. Molding of the side port 128 having the fluid directing ramp 134 is also simplified as compared to molding of an offset side port, as the parting line can be uniform along the needle free connector 100, and creation of the fluid directing ramp 134 only involves a minor modification to the core pin geometry used to form the side port 128.

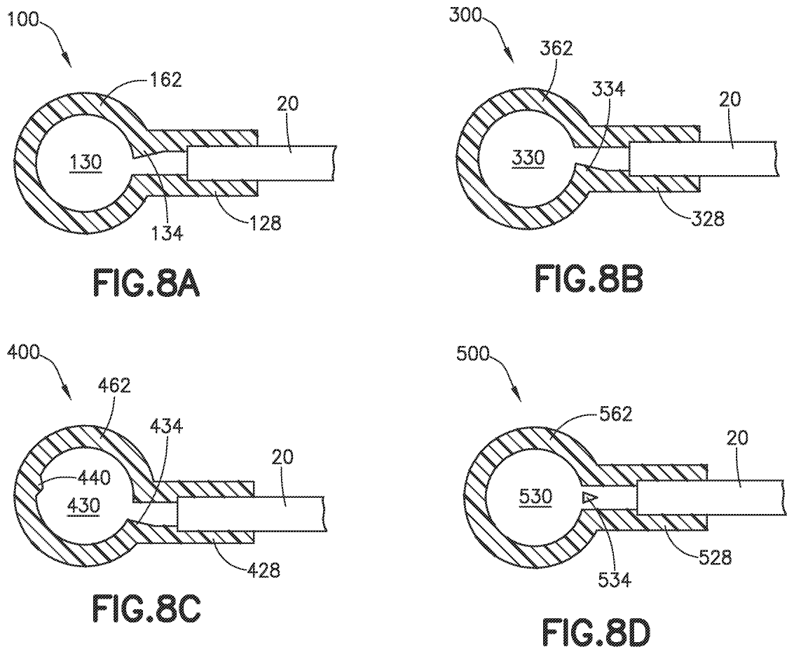

Referring to FIGS. 8A-8D, various side port flow diversion concept in accordance with aspects of the present disclosure are illustrated. FIG. 8A shows the needle free connector 100 described above with respect to FIGS. 6 and 7, wherein a fluid directing ramp 134 is provided on an upper surface of the side port 128 to divert flow downward relative to the central plane of main cavity portion 130, with the side port 128 not offset relative to the main cavity portion 130. However, referring to FIG. 8B, in another embodiment, a needle free connector 300 may be provided with a body 362 and a side port 328 directed into a main cavity portion 330. The side port 328 may include a fluid directing ramp 334 positioned on a lower surface thereof in order to divert flow upward relative to the central plane of the main cavity portion 330. Accordingly, it is to be understood that the side port flow diversion concepts described herein are not limited to directing fluid in only one direction relative to the main cavity portion of the needle free connector.

Referring to FIG. 8C, a needle free connector 400 in accordance with another aspect of the present disclosure is shown. Unlike needle free connectors 100, 300 described above, needle free connector 400 may include a side port 428 extending from a body 462, with the side port 428 being offset from the central plane of a main cavity portion 430. While the offset positioning of the side port 428 may aid in encouraging a swirling flow of fluid forced therethrough, the side port 428 may further include a fluid directing ramp 434 configured to further direct fluid toward a proximal flow diverter 440 within the main cavity portion 430 of the needle free connector 400. While FIG. 8C illustrates the fluid directing ramp 434 being positioned on a lower surface of the side port 428 in order to divert flow upward relative to the central plane of the main cavity portion 430, it is to be understood that fluid directing ramp 434 may alternatively be positioned an on upper surface of the side port 428.

FIG. 8D illustrates a needle free connector 500 according to another aspect of the present disclosure. Needle free connector 500 includes a body 562 defining a main cavity portion 530, with a side port 528 extending from the body 562. Within the side port 528 is a flow splitter 534, which is configured to split the fluid flow entering the main cavity portion 530 from the side port 528, thereby encouraging a turbulent flow within the main cavity portion 530 to aid in flushing of the needle free connector 500. As shown in FIG. 8D, the side port 528 is not offset relative to the main cavity portion 530. However, in alternative embodiments, it is to be understood that side port 528 may be offset relative to main cavity portion 530.

Although described with respect to needle free connectors used with integrated intravenous catheters, it is to be understood that the concepts described herein may be applicable to any medical device fluid junction having fluid inlets or outlets with different central axes. The fluid junction may be an optimization of one or more of side fluid path entrance angle, central or planar offset, side port flow directing ramp(s), and/or optimized position of proximal flow diverting feature(s). For example, catheter adapters may include the concepts described herein, wherein a first port may be coupled to a catheter, a second port may include a non-luer accessible septum, and a side port is provided having a different central axes to the first port and the second port.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. An integrated intravenous catheter comprising:
a catheter adapter comprising a catheter and an inlet, the catheter configured to be inserted into a patient's vasculature;
a needle free connector comprising a first port, a second port positioned opposite the first port, and a side port positioned between the first port and the second port, wherein the needle free connector further comprises a main cavity portion having a tapered sidewall and a distal taper portion, and wherein the tapered sidewall gradually narrows in a direction of the distal taper portion;
intermediate tubing extending between the inlet of the catheter adapter and the first port of the needle free connector; and
extension tubing extending from the side port of the needle free connector.

2. The integrated intravenous catheter of claim 1, wherein the needle free connector comprises a body defining a flow path extending between the first port and the second port.

3. The integrated intravenous catheter of claim 2, wherein the side port is offset from a center of the flow path.

4. The integrated intravenous catheter of claim 2, wherein the side port is not offset from a center of the flow path.

5. The integrated intravenous catheter of claim 2, wherein the body of the needle free connector further comprises a lip portion extending 360° around the needle free connector, and wherein the lip portion is configured to provide a latching surface for engagement with clip portions of a lock of a blood draw device when coupled to the needle free connector.

6. The integrated intravenous catheter of claim 5, further comprising a gap formed between the lip portion and the side port.

7. The integrated intravenous catheter of claim 1, further comprising a medical connector positioned at an end of the extension tubing.

8. The integrated intravenous catheter of claim 1, wherein the needle free connector comprises a body defining a longitudinal axis extending between the first port and the second port, and wherein the side port extends from the body at an angle of 15-165 degrees relative to the longitudinal axis of the body.

9. The integrated intravenous catheter of claim 1, wherein the main cavity portion of the needle free connector comprises an internal structure configured to redirect fluid when fluid enters the needle free connector via the side port.

10. The integrated intravenous catheter of claim 1, wherein the side port comprises a fluid flow path, and wherein an internal surface of the side port at a distal portion of the fluid flow path comprises a fluid directing ramp.

11. The integrated intravenous catheter of claim 10, wherein the fluid directing ramp is positioned on a lower internal surface of the side port and is configured to divert flow upward relative to a central plane of the main cavity portion of the needle free connector.

12. The integrated intravenous catheter of claim 10, wherein the fluid directing ramp is positioned on an upper internal surface of the side port and is configured to divert flow downward relative to a central plane of the main cavity portion of the needle free connector.

13. The integrated intravenous catheter of claim 1, wherein the side port comprises a fluid flow path, and wherein a distal portion of the fluid flow path of the side port comprises a flow splitter.

14. A needle free connector comprising:
a body;
a first port positioned at a first end portion of the body;
a second port positioned at a second end portion of the body opposite the first port; and
a side port positioned between the first port and the second port,
wherein the body comprises a main cavity portion positioned between the first port and the second port, the main cavity portion having a tapered sidewall and a distal taper portion, and wherein the tapered sidewall gradually narrows in a direction of the distal taper portion.

15. The needle free connector of claim 14, wherein the body further comprises a lip portion extending 360° around the needle free connector, and wherein the lip portion is configured to provide a latching surface for engagement with clip portions of a lock of a blood draw device when coupled to the needle free connector.

16. The needle free connector of claim 15, further comprising a gap formed between the lip portion and the side port.

17. The needle free connector of claim 14, wherein the side port comprises a fluid flow path, and wherein an internal surface of the side port at a distal portion of the fluid flow path of the side port comprises a fluid directing ramp.

18. The needle free connector of claim 17, wherein the fluid directing ramp is positioned on a lower internal surface of the side port and is configured to divert flow upward relative to a central plane of the main cavity portion of the needle free connector.

19. The needle free connector of claim 17, wherein the fluid directing ramp is positioned on an upper internal surface of the side port and is configured to divert flow downward relative to a central plane of the main cavity portion of the needle free connector.

20. The needle free connector of claim 14, wherein the side port comprises a fluid flow path, and wherein a distal portion of the fluid flow path of the side port comprises a flow splitter.
